(19) United States
(12) Patent Application Publication
(10) Pub. No.: US 2026/0216235 A1
(43) Pub. Date: Jul. 30, 2026

(54) AN ANTIMICROBIAL GEL COMPOSITION

(71) Applicant: BACTIGUARD AB, Tullinge (SE)

(72) Inventors: Ranjeni KRISHNEN, Penang (MY); Sathish Komar SUBRAMANIAM, Penang (MY); Chee Keong KOH, Penang (MY); Billy SÖDERVALL, Markaryd (SE)

(21) Appl. No.: 19/147,273

(22) PCT Filed: Jan. 19, 2024
(86) PCT No.: PCT/EP2024/051312

§ 371 Date: Jul. 10, 2025

(30) Foreign Application Priority Data
Jan. 20, 2023 (SE) 2350050-7

(51) Int. Cl. A61K 33/00; A61K 9/08; A61K 47/02; A61P 17/02; A61P 31/00

(52) U.S. Cl. CPC A61K 33/00; A61K 9/08; A61K 47/02; A61P 17/02; A61P 31/00

(57) ABSTRACT

There is provided an antimicrobial composition for wound management and a method of producing it. The antimicrobial composition comprises sodium hypochlorite (NaOCl) in an amount in the range 0.015-0.15 wt %, hypochlorous acid (HOCl) in an amount in the range 0.002-0.1 wt %, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is 0.15 or less.

### FIELD OF INVENTION

The present invention relates to an antimicrobial composition, a method for producing said antimicrobial composition, and a valve system apparatus as well as a method for wound management comprising said antimicrobial composition.

### BACKGROUND OF THE INVENTION

In general, wounds are a hidden epidemic among the world's population, with significant social and economic consequences that impair millions of people's quality of life. While acute wound management in a healthy patient is relatively manageable, patients with chronic wounds can be difficult to assess and maintain. Cases of chronic wounds are on the rise, yet existing standard of care solutions fail to meet treatment principles. While bandages and gauzes are useful for reducing bleeding, they have a number of drawbacks. They are not biodegradable, are prone to infection, and are unsuitable for wounds with uneven shapes. They have the potential to cause secondary tissue destruction and are ineffective for wound healing. Antibiotic resistance, as well as a weak immune response, creates challenges in the treatment of chronic infections. Owing to this, extensive studies have been carried out on the topic of wound healing processes and wound management devices. Existing research recognizes the critical role played by hydrogels in wound healing. The supply of moisture to the wound, allowing for painless debridement of necrotic and infected tissue, as well as granulation and complete healing. However, given present hydrogels have a high water content, they are not totally absorbent, therefore they are best suited for wounds with light to moderate exudation. In line with this, an antimicrobial is needed to combat infection and to assist in wound healing process. Therefore, in light of the foregoing discussion, there exists a need to overcome the aforementioned drawbacks associated with the currently available antimicrobial hydrogels.

US Patent Application 20200138953A1 discloses a stabilized hypohalous acid solution, particularly a stabilized solution or formulation comprising hypobromous acid and a stabilizing amount dissolved inorganic carbon in the form of bicarbonate or carbonate of alkaline earth metals.

US Patent Application 20200138953A1 discloses a bag-on-valve can with a hypochlorous acid solution and a chlorine stabilizer which is suitable to be used in disinfecting food processing surfaces and sanitizing medical equipment in hospitals.

CN 113069413 A discloses a hypochlorous acid gel supporting electron beam irradiation sterilization. The hypochlorous acid gel is prepared from the following raw materials in parts by mass: 50 to 70 parts of a gel matrix, 27.4 to 49.979 parts of a phosphate buffer solution, 0.001 to 0.6 part of hypochlorous acid and 0.02 to 2 parts of sodium hypochlorite, wherein the phosphate buffer solution is prepared from boric acid, potassium dihydrogen phosphate, sodium chloride and pure water. The hypochlorous acid gel has a neutral pH value, and is said to have no irritation to a wound surface. Preferably, the pH of the hypochlorous acid gel is 5.5 to 8.0. The only example has pH 6.8. The gel matrix powder is preferably any one or more of magnesium lithium silicate, magnesium aluminum silicate and magnesium sodium fluorosilicate. It should also be noted that a phosphate buffer solution is mandatory and that the gel thus has a certain buffer capacity β with regard to the pH value, which buffer capacity β is fairly high. The buffer capacity according to the formulation in the example of CN 113069413 has been calculated by the us to β=0.189≈0.19≈0.2.

For compositions in the prior art comprising sodium hypochlorite and hypochlorous acid it has turned out that the shelf life is often not satisfying for a convenient use.

Further, it is desirable to improve the treatment of wounds by providing additional delivery forms of the antibacterial agent(s).

It is a problem in the art that antimicrobial compositions are irritating to the skin. For hypochlorite and hypochlorous acid the optimum pH value for the antimicrobial effect is at an acidic pH such as for instance pH 3-7. At the same time an acidic pH is more irritant to the skin and can give the user a discomforting feeling.

### SUMMARY OF THE INVENTION

One object of the present invention is to obviate at least some of the disadvantages in the prior art and provide an improved antibacterial composition in gel form.

In a first aspect there is provided an antimicrobial composition, comprising sodium hypochlorite (NaOCl) in an amount in the range 0.015-0.15 wt %, hypochlorous acid (HOCl) in an amount in the range 0.002-0.1 wt %, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm and wherein the composition has a pH in the interval 7.5-10.0.

In a second aspect there is provided a method (400) for manufacturing an antimicrobial composition, the method comprising the steps of:
a) providing
   i. purified water, wherein the purified water before addition to the composition has a hardness measured according to ISO 15923-2:2017 of less than 5 ppm and a resistivity measured according to ISO 3696:1987 of more than 15 MΩ/cm;
   ii. a synthetic silicate clay based thickening agent;
   iii. a sodium hypochlorite solution, and
   iv. sodium chloride, and
b) mixing the purified water, the synthetic silicate clay based thickening agent, the sodium hypochlorite solution, and the sodium chloride to obtain the antimicrobial composition,
wherein the composition has a pH in the interval 7.5-10.0.

In a third aspect there is provided a valve system apparatus for wound management, said valve system apparatus comprising, a bag-on-valve (BoV) inside a pressurized storage canister; and an antimicrobial composition for wound management contained in said bag, said antimicrobial composition comprising sodium hypochlorite (NaOCl) in an amount in the range 0.015-0.15 wt %, hypochlorous acid (HOCl) in an amount in the range 0.003-0.03 wt %, a synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is 0.15 or less.

In a fourth aspect there is provided a method for producing a valve system apparatus for wound management comprising steps of:
a) placing a bag-on-valve (BoV) inside a storage canister;
b) crimping the BoV onto the storage canister followed by filling said storage canister with propellant gas to obtain a pressurized storage container; and
c) filling the bag of the BoV with the antimicrobial composition as described above via the valve of the BoV.

In a fifth aspect there is provided a method for delivery of an antimicrobial composition to a wound, said method comprising the step of applying an antimicrobial composition to a wound, said antimicrobial composition comprising sodium hypochlorite (NaOCl) in an amount in the range 0.015-0.15 wt %, hypochlorous acid (HOCl) in an amount in the range 0.003-0.03 wt %, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is 0.15 or less.

In a sixth aspect there is provided a method for the treatment of a wound, wherein there is applied to the wound an antimicrobial composition comprising sodium hypochlorite (NaOCl) in an amount in the range 0.015-0.15 wt %, hypochlorous acid (HOCl) in an amount in the range 0.002-0.1 wt %, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is 0.15 or less.

In a seventh aspect there is provided a composition comprising sodium hypochlorite (NaOCl) in an amount in the range 0.015-0.15 wt %, hypochlorous acid (HOCl) in an amount in the range 0.002-0.1 wt %, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is 0.15 or less for prevention or treatment of infections in wounds.

Further embodiments of the present invention are defined in the appended dependent claims.

These and other aspects of the embodiments herein will be better appreciated and understood when considered in conjunction with the description and the accompanying drawings. It should be understood, however, that the following descriptions, while indicating certain embodiments and numerous specific details thereof, are given by way of illustration and not of limitation. Many changes and modifications may be made within the scope of the embodiments herein without departing from the spirit thereof, and the embodiments herein include all such modifications.

An advantage of the invention is that a pain sensation on the skin can be suppressed at the same time as there is a boost in the antimicrobial effect locally in an open wound where it is needed. This is due to the low buffer capacity. The pH is a bit higher than neutral, i.e. above pH 7.5 and is thereby less irritant to the skin. In a wound the pH is typically lower than 7.5 and due to the low buffer capacity of the antimicrobial gel, the pH of the antimicrobial gel will be lower than 7.5 when mixed with the fluid in the wound. Because of the lower pH the antimicrobial effect will then be higher locally in the wound. Thus the enhanced antimicrobial effect is located where needed, i.e. in the wound. At the same time the skin is not irritated by a low pH.

It is an advantage of the invention that the antimicrobial composition is provided in gel form, which makes additional treatment methods possible compared to an aqueous solution, which is not in gel form. A gel can be applied for instance to a wound in the skin and will remain at the skin with a certain thickness and while being moist for a while so that it can exert its antimicrobial action for an extended period of time.

The shelf life is greatly improved, which is an advantage compared to many other compositions comprising sodium hypochlorite and hypochlorous acid, in particular in gel form. For many similar compositions in the prior art a shorts shelf life is a problem.

### BRIEF DESCRIPTION OF THE DRAWINGS

Other objects, features, and advantages of the invention will be apparent from the following description when read with reference to the accompanying drawings. In the drawings, wherein like reference numerals denote corresponding parts throughout the several views:

FIG. 1 depicts a table illustrating the composition of each ingredient of the antimicrobial composition of the present technology, in accordance with an embodiment. The active ingredient composition is determined by Iodometric Titration Method following ISO 7393-3: 1990 (E) Water Quality—Determination of Free Chlorine and Total Chlorine—Part 3: Iodometric Titration Method for the Determination of Total Chlorine.

FIG. 2 depicts test results of preservative test according to USP preservative efficacy test, in accordance with an exemplary scenario. USP 41 NF 36, Chapter 51 method is referred in conducting the antimicrobial effectiveness challenge test.

FIG. 3 depicts test results of biocompatibility of the antimicrobial composition of the present technology as per ISO 10993 Biological evaluation of medical devices for in vivo and in vitro studies, in accordance with an exemplary scenario. The biocompatibility testing including cytotoxicity, sensitization, intracutaneous reactivity, acute systemic toxicity and material mediated pyrogen tests were conducted.

FIG. 4 is a flow diagram depicting the steps involved in the method of producing the antimicrobial composition of the present technology, in accordance with an embodiment.

FIG. 5 provides an example of the process of packaging the antimicrobial composition of the present invention into a valve system apparatus comprising BoV.

### DETAILED DESCRIPTION OF THE INVENTION

Before the invention is disclosed and described in detail, it is to be understood that this invention is not limited to particular configurations, process steps and materials disclosed herein as such configurations, process steps and materials may vary somewhat. It is also to be understood that the terminology employed herein is used for the purpose of describing particular embodiments only and is not intended to be limiting since the scope of the present invention is limited only by the appended claims and equivalents thereof.

It must be noted that, as used in this specification and the appended claims, the singular forms “a”, “an” and “the” include plural referents unless the context clearly dictates otherwise.

The following terms are used throughout the description and the claims.

“Antimicrobial” as used herein is the property of suppressing or eliminating microbial growth. Microbial growth includes but is not limited to bacterial growth.

The buffer capacity as used herein is a quantitative measure of the resistance to change of pH of a solution containing a buffering agent with respect to a change of acid or alkali concentration. It can be defined as

$$β = \frac{d ( C )}{d ( pH )} ,$$

wherein d(C) is an infinitesimal amount of added base, or added acid and where d(pH) is an infinitesimal change in pH.

The buffer capacity is dependent on the pH. For a weak acid the buffer capacity rises to a local maximum at pH=pKₐ. The height of this peak depends on the value of pKₐ. Buffer capacity is negligible when the concentration [HA] of buffering agent is very small and increases with increasing concentration of the buffering agent.

The buffer capacity β of the present gel should be less than 0.15. The buffer capacity is measured at pH 8.0. A lower buffer capacity is generally better since the pH in an open wound comprising a liquid and with a pH which is lower than the pH of the gel will decrease more. This in turn will give a more pronounced antimicrobial activity. Thus a lower buffer capacity is generally better than a higher buffer capacity. The upper limit of the buffer capacity β=0.15 should be seen as an upper limit and so that a lower buffer capacity is even better. Thus the buffer capacity can in different embodiments be lower than β=0.15, 0.14, 0.13, 0.12, 0.11, 0.10, 0.090, 0.080, 0.070, 0.060, 0.050, 0.040, 0.030, 0.020, and 0.010. The buffer capacity is measured at pH 8.0. In one embodiment the upper limit for the buffer capacity is such that it is the buffer capacity of the ingredients according to claim 1, i.e. without added buffer. In an alternative embodiment the limit for the buffer capacity is replaced with a restriction that no buffer is added in addition to the ingredients according to claim 1.

The present buffer capacity of 0.15 or less is lower than the buffer capacity in CN 113069413, which is about 0.19. Thereby the pH of the present gel changes more in a wound so that the pH becomes more as in the wound, which is typically lower than pH 7.5. At a lower pH the composition is more antimicrobial.

In the below table the buffer capacity of CN 113069413 and the content of the present formulation is investigated. For the present gel the contents are shown for the minimum and maximum amounts of the ingredients.

| | CN | Present | Present |
|---|---|---|---|
| | 113069413 | gel min | gel Max |
| | wt % | wt % | wt % |
| Magnesium Lithium | 4.000 | 1.000 | 10.000 |
| Silicate | | | |
| Pure Water | 52.538 | 98.953 | 89.450 |
| Phosphate Buffer | | | |
| Boric Acid diphosphate | 1.600 | | |
| Potassium Hydrogen | 1.600 | | |
| Sodium Chloride | 0.200 | | |
| Pure Water | 40.000 | | |
| Sodium Chloride | 0.000 | 0.030 | 0.300 |
| Hypochlorous Acid | 0.002 | 0.002 | 0.100 |
| Sodium Hypochlorite | 0.060 | 0.015 | 0.150 |
| | 100.000 | 100.000 | 100.000 |

Using the Henderson-Hasselbalch Equation, the above numbers were redefined/theoretically calculated using the formula below.

$$pH = pKa + Log ( A ) / ( HA )$$

| | | CN | Present | Present |
|---|---|---|---|---|
| | | 113069413 | gel min | gel Max |
| Sodium | pKa | 7.52 | 7.52 | 7.52 |
| Hyphochlorite | | | | |
| Base | (A) | 0.2 | 0.03 | 0.3 |
| Concentration | | | | |
| Acid | (HA) | 1.602 | 0.002 | 0.1 |
| Concentration | | | | |

| | | | |
|---|---|---|---|
| pH Estimated | 6.62 | 8.70 | 8.00 |

The buffer capacity for CN 113069413 is calculated with the Boric Acid molarity as below:

Using the β = n/ΔpH

| | |
|---|---|
| Boric Acid Molarity | 0.0378 |
| Buffer Limits for D1 β | 0.189 |

It can clearly be seen that the buffer capacity of the present gel has to be considerably lower than the buffer capacity of CN 113069413, since CN 113069413 comprises a phosphate buffer, which the present gel does not comprise.

Based, on the calculation above, it is estimated the buffer capacity for the D1 is at 0.189.

In the following detailed description, numerous specific details are set forth in order to provide a thorough understanding of the invention. However, it will be understood by those of ordinary skill in the art that the invention may be practiced without these specific details. In other instances, well-known methods, procedures and/or components have not been described in detail so as not to obscure the invention.

In the first aspect there is provided an antimicrobial composition, comprising sodium hypochlorite (NaOCl) in an amount in the range 0.015-0.15 wt %, hypochlorous acid (HOCl) in an amount in the range 0.002-0.1 wt %, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity measured at pH 8.0 is 0.15 or less.

One advantage of the present invention is that the composition increases the moisture within the wound, which makes the composition effective in assisting the debridement and de-sloughing process in dry necrotic wounds. The composition is intended for moistening and debridement of acute and chronic wounds such as stage I-IV ulcers, venous stasis and diabetic ulcerations, post-surgery wounds, first and second-degree burns, and bed sores. The antimicrobial composition of the present technology helps maintain a moist wound environment, promote granulation and epithelialization, and facilitate autolytic debridement. The composition has antimicrobial properties. The sodium hypochlorite provides bacteriostatic properties. The antimicrobial composition is a clear composition that helps maintain a moist wound environment that is conducive to wound healing.

It has turned out that various additives will impair the shelf life of compositions comprising sodium hypochlorite and hypochlorous acid. For instance, it has turned out that various impurities as well as additives in the water used for the preparation of the composition can speed up the degradation of the active antimicrobial substances so that the activity of the composition decreases and so that the shelf life becomes unacceptably short. Although the detailed reaction mechanism is not known on a molecular level, the inventors have found that a high water hardness and the presence of metal ions will accelerate the degradation of the active compounds.

The water hardness is measured according to ISO 15923-2:2017 as the total hardness and is presented in ppm. In one embodiment, the hardness does not exceed 5 ppm. In one embodiment, the hardness does not exceed 4 ppm. In one embodiment the hardness does not exceed 3 ppm. In one embodiment the hardness does not exceed 2 ppm. In one embodiment the hardness does not exceed 1 ppm.

The content of metal ions is measured by measuring the resistivity of the water. This is a good approximation since positive counter-ions most often are metal ions and since also H⁺ is assumed to contribute to the degradation of the active ingredients. The conductivity of the water, (i.e. the inverse of the resistivity) is measured as outlined in ISO 3696:1987 and then the resistivity is calculated as the inverse. The resistivity is in one embodiment higher than 15 MΩ/cm. In one embodiment the resistivity is higher than 16 MΩ/cm. In one embodiment the resistivity is higher than 17 MΩ/cm. In one embodiment the resistivity is higher than 17.2 MΩ/cm. In one embodiment the resistivity is higher than 17.5 MΩ/cm.

In particular the inventors believe that the combination of a high water hardness and the presence of metal ions contribute to an accelerated degradation of the active components. Thus, it is important that both the amount of metal ions and the water hardness are kept low for the water used for the preparation of the composition. When the water hardness and the resistivity are kept within the above boundaries (i.e. resistivity higher than 15 MΩ/cm and hardness below 5 ppm), the shelf life of the composition improves.

In addition, also other quality criteria are suitable for the water used in the preparation of the product, not least since the composition is a medical product and should fulfil high standards.

The TOC is measured according to ISO 20236:2018 and recalculated to ppb by weight. The TOC does in one embodiment not exceed 50 ppb. In one embodiment, the TOC is less than 10 ppb. All calculated by weight.

TDS as measured according to ASTM D5907-10 does in one embodiment not exceed 50 ppm by weight. In one embodiment, the TDS does not exceed 15 ppm. In another embodiment, the TDS does not exceed 5 ppm. All calculated by weight.

Further, the levels of heavy metals should be low. In one embodiment, the amount of the heavy metals arsenic, cadmium, and lead are together lower than 0.1 ppm by weight.

Actually, it has turned out that the selection of the thickener is not trivial since it should be both acceptable from a medical point of view and it should also not accelerate the decomposition of the active antimicrobial components. All additives to the composition have the potential of accelerating the decomposition of the active ingredients and thus all additives have to be carefully selected so that they do not accelerate the decomposition of the antimicrobial compounds in the composition. It has turned out that the selected synthetic silicate clay based thickening agent fulfils these requirements. In particular, the antimicrobial ingredients are not degraded when a synthetic silicate clay based thickening agent is used and when the amount of metal ions and water hardness is within the required boundaries.

The use of a synthetic silicate clay based thickening agent together with use of water with a resistivity above 15 MΩ/cm and a hardness below 5 ppm gives an improved shelf life by not accelerating the decomposition of the antimicrobial agents.

All above limits for the water applies to the purified water before the addition of the water to the composition.

In one embodiment, the amount of sodium hypochlorite is in the range 0.04-0.06 wt %.

In one embodiment, the amount of hypochlorous acid (HOCl) is in the range 0.005-0.02 wt %. This range of NaOCl and HOCl concentrations shown good biocompatibility and efficacy against microorganisms.

In one embodiment, the amount of sodium chloride is in the range 0.05-0.2 wt %. This range of NaCl is suitable to act as stabilizer and preservative ingredients.

In one embodiment, the synthetic silicate clay based thickening agent comprises lithium magnesium sodium silicate.

In one embodiment, the synthetic silicate clay based thickening agent is present in an amount in the interval 3-5 wt %. This synthetic silicate clay is found to be the suitable to retain the active ingredients in semi-liquid form. Other thickeners such as dimethicone, carboxy methyl cellulose, Xanthan Gum and carbomer was not able to retain the active ingredients and did not produce the desired gel.

In one embodiment, purified water constitutes the remaining part of the composition in addition to sodium hypochlorite (NaOCl), hypochlorous acid (HOCl), synthetic silicate clay based thickening agent, and sodium chloride (NaCl). Purified water is needed to ensure there is no other ions present in the gel that can disrupt the active ingredients' stability.

The composition has a pH above 7.5. In one embodiment, the composition has a pH in the range of 7.5 to 10.0. The growth of wound healing cells including fibroblasts and keratinocytes is facilitated by an alkaline pH (Teshima et al, 2020). In an alternative embodiment, the composition has a pH in the range of 7.5 to 9.5. In an alternative embodiment, the composition has a pH in the range of 7.5 to 9.0. In an alternative embodiment, the composition has a pH in the range of 8.0 to 9.0. In an alternative embodiment, the composition has a pH in the range of 8.0 to 9.5 In an alternative embodiment, the composition has a pH in the range of 8.0 to 10.0 For the invention in general including the description, examples and the claims, the pH is measured following the method described in ASTM E70-19.

In relation to the prior art including CN 113069413 A, the prior art the present invention has a much lower buffer capacity. It is known that the intensity of the pain sensation is much lower at a high pH compared to a lower pH. (Amiloride-blockable acid-sensing ion channels are leading acid sensors expressed in human nociceptors, Shinya Ugawa, J. Clin. Invest. 110:1185-1190 (2002). doi:10.1172/JCI200215709.). Thus it is suitable to have a higher pH than neutral with regard to pain sensation. However the pH becomes lower in a wound with lower pH due to the low buffer capacity. A wound typically has a lower pH than 7.5. This gives a higher antimicrobial effect locally in the wound.

The hypochlorous acid (HOCl) is present in an equilibrium so that at a very low pH, below about 1.5, Cl₂ is the dominating species, between about pH 1.5-7.5 the HOCl is the dominating species, and above pH 7.5, the (OCl)⁻ is dominating species. The hypochlorous acid (HOCl) is present in different form depending on the pH, although it is present mainly as (OCl)⁻ in the present invention at a pH above 7.5 it is nevertheless referred to as hypochlorous acid (HOCl). It is further known that the species HOCl, which is the dominant species at about pH 1.5-7.5 is the most antibacterial compared to (OCl)⁻. In view of this it is natural that many antibacterial compositions involving hypochlorous acid (HOCl) have a pH in the pH range 1.5-7.5. Because of the low buffer capacity of the gel, the gel will mix with fluids in a wound so that the pH becomes lower and the HOCl becomes the dominating species with a higher antimicrobial effect locally in the wound. Normally wounds have a lower pH than 7.5. Thus the composition is particularly suitable for treating wounds which have neutral or acidic pH.

The pH of the present invention above 7.5 provides sufficient antimicrobial activity for the wound treatment. At the same time there is an additional effect providing additional antimicrobial activity. Most wounds have a neutral pH and for an open wound the composition of the present invention will be mixed with liquid in the open wound and then the pH of the mixture will be lower than 7.5. This lowering of the pH will improve the antimicrobial effect of the composition. It should be noted that this effect is present in the wound and very close to the wound where the composition is mixed with fluid from the wound. This give an advantage of a local boost of the antimicrobial effect right where it is desired, i.e. in and/or close to the wound. Further away from the open wound the composition according to the present invention will be less irritating to the skin because of the higher pH value. Thus the invention combines a lower irritation with an antimicrobial effect where needed.

It should be noted that the buffer capacity in the composition according to the present invention is low, so that the pH value is affected by fluid in an open wound. In relation to this it should be noted that CN 113069413 comprises a phosphate buffer so that the pH in an open wound would not change to a noticeable extent. Thus it is an advantage that the composition according to the present invention does not comprise a buffer.

In one embodiment, the viscosity of the composition is in the range 8000-10000 cp. This high viscosity enables the gel to stay in the cavity of wound and provide more moisture. The viscosity is measured using Brookfield viscometer according to ISO 2555:2018 Plastics—Resins in the liquid state or as emulsions or dispersions—Determination of apparent viscosity using a single cylinder type rotational viscometer method.

In the second aspect there is provided a method (400) for manufacturing an antimicrobial composition, the method comprising the steps of:
a) providing
   i. purified water, wherein the purified water before addition to the composition has a hardness measured according to ISO 15923-2:2017 of less than 5 ppm and a resistivity measured according to ISO 3696:1987 of more than 15 MΩ/cm;
   ii. a synthetic silicate clay based thickening agent;
   iii. a sodium hypochlorite solution, and
   iv. sodium chloride, and
b) mixing the purified water, the synthetic silicate clay based thickening agent, the sodium hypochlorite solution, and the sodium chloride to obtain the antimicrobial composition,
wherein the composition has a pH in the interval 7.5-10.0, and wherein the buffer capacity β measured at pH 8.0 is 0.15 or less.

In one particular embodiment of the second aspect the method comprises the steps of:
a) filling (402) purified water into a container;
b) adding (406) Lithium Magnesium Sodium Silicate during mixing and stirring (408) at least 90 seconds;
c) adding (410) a sodium hypochlorite solution and stirring at least 30 seconds; and
d) adding (412) sodium chloride (NaCl) to the mixture and stirring for at least 120 seconds.

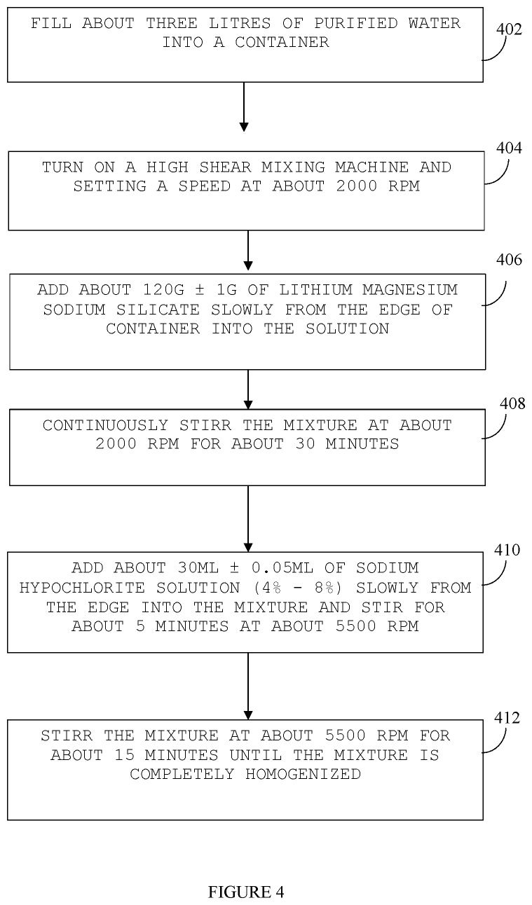

This particular embodiment is depicted in FIG. 4. In one embodiment, the steps b) to d) in FIG. 4 are performed in sequential order.

In one embodiment, the sodium hypochlorite solution in step c) has a concentration in the range 4-8 wt %.

In the third aspect there is provided a valve system apparatus for wound management, said valve system apparatus comprising, a bag-on-valve (BoV) inside a pressurized storage canister; and an antimicrobial composition for wound management contained in said bag, said antimicrobial composition comprising sodium hypochlorite (NaOCl) in an amount in the range 0.015-0.15 wt %, hypochlorous acid (HOCl) in an amount in the range 0.003-0.03 wt %, a synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is 0.15 or less.

In one embodiment, the pressurized storage canister comprises a non-flammable propellant.

In one embodiment, said pressurized storage canister is an aerosol spray canister.

In the fourth aspect there is provided a method for producing a valve system apparatus for wound management comprising steps of:
a) placing a bag-on-valve (BoV) inside a storage canister;
b) crimping the BoV onto the storage canister followed by filling said storage canister with propellant gas to obtain a pressurized storage container; and
c) filling the bag of the BoV with the antimicrobial composition as described above via the valve of the BoV.

In one embodiment, said steps a) to c) of said method for producing a valve system apparatus for wound management are performed in a sequential manner.

In the fifth aspect there is provided a method for delivery of an antimicrobial composition to a wound, said method comprising the step of applying an antimicrobial composition to a wound, said antimicrobial composition comprising sodium hypochlorite (NaOCl) in an amount in the range 0.015-0.15 wt %, hypochlorous acid (HOCl) in an amount in the range 0.003-0.03 wt %, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is 0.15 or less.

Various embodiments of the present invention provide an antimicrobial composition for wound management and a method for producing the antimicrobial composition. The antimicrobial composition comprises sodium hypochlorite (NaOCl), hypochlorous acid (HOCl), synthetic silicate clay based thickening agent, sodium chloride (NaCl) and purified water, wherein the purified water has to fulfil certain criteria for the hardness and resistivity.

In the sixth aspect there is provided a method for the treatment of a wound, wherein there is applied to the wound an antimicrobial composition comprising sodium hypochlorite (NaOCl) in an amount in the range 0.015-0.15 wt %, hypochlorous acid (HOCl) in an amount in the range 0.002-0.1 wt %, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is 0.15 or less.

In the seventh aspect there is provided a composition comprising sodium hypochlorite (NaOCl) in an amount in the range 0.015-0.15 wt %, hypochlorous acid (HOCl) in an amount in the range 0.002-0.1 wt %, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is 0.15 or less.

for prevention or treatment of infections in wounds.

Thus, the sixth and sevenths aspects provide treatment of wounds and in particular prevention and/or treatment of infections in wounds.

The antimicrobial composition of the present technology has a pH above 7.5 and a free available chlorine (30 ppm-200 ppm) and will be able to kill wide range of microorganism including bacteria and fungi. In one embodiment, free available chlorine as measured according to ISO 7393-2:2017 is in the range 30-200 ppm. The free available chlorine provides the composition the ability to eliminate a wide range of microorganism including bacteria and fungi.

In an embodiment, the antimicrobial composition of the present invention is a hemostatic agent. In an embodiment, the antimicrobial composition of the present invention is a chemical hemostatic agent. Said antimicrobial composition when applied promotes hemostasis is able to stop or slow down bleeding of a wound.

In an embodiment of the invention, a valve system for wound management is provided. Said valve system comprises or consists of a bag inside a pressurized storage canister, and an antimicrobial composition for wound management contained in said bag. Said antimicrobial composition comprises sodium hypochlorite (NaOCl), hypochlorous acid (HOCl), synthetic silicate clay based thickening agent, sodium chloride (NaCl) and purified water. In an embodiment, the antimicrobial composition of the invention is a when stored inside said valve system apparatus. In another embodiment, the antimicrobial composition of the invention is an aerosol-sprayed composition when sprayed out of said valve system apparatus or when applied to a wound via said valve system apparatus. In one embodiment, said antimicrobial composition of the invention when applied from said valve system apparatus to a wound, said antimicrobial composition becomes aerated and becomes a foam-like or froth-like sprayed composition over a wound. In an embodiment, said bag containing an antimicrobial composition for wound management is free of propellant, or chemical-propellant-free. The present invention of a valve apparatus does not utilize a chemical propellent having in contact with the antimicrobial composition of the present invention.

In one embodiment, said pressurized storage canister of the valve system apparatus of the invention comprises a chemical propellant. Particularly, the present invention does not use any flammable material, or any flammable propellant, which also provides a reduced risk when handing said valve system apparatus when exposed to high temperatures or ignition. The present invention's valve system apparatus not using any flammable material also enables prolonged and preserved shelf-life as flammability is not a concern especially when said valve system apparatus is stored.

The chemical propellant comprised in the pressurized storage canister provides pressure to the bag inside the pressurized storage canister, which allow the composition inside said bag to be indirectly propelled outside the valve system apparatus when the composition is being applied. As the chemical propellant is contained outside the bag, the separation of the chemical propellant to the bag prevents any change in the quality or properties of the antimicrobial composition stored in said bag. In one embodiment, said chemical propellant is nitrogen gas. Using nitrogen gas as propellant is not only non-flammable, inert, extremely low boiling point (−195° C.), but it also helps maintain flow of the most viscous liquids through piping, which allows dispensing of near 99.9 wt % of compositions stored in the bag

In an embodiment, the pressurized storage canister, used in said valve system apparatus of the invention, is an aerosol spray container suitable to be used to spray foams or foam-like form of compositions. The utility of a valve system in the apparatus of the present invention allows the product to be dispensed in pure form i.e. without any further mixed unnecessary components, allowing storage via said valve system to receive more composition amounts compared to traditional and pre-existing bag-on-valve product packaging. Furthermore, the valve system of the present invention allows the dispensing of the stored antimicrobial composition at 99.99 wt % preventing any unused remaining composition. The present valve system apparatus may also be used in continuous and uninterrupted spraying in any angle and even/controlled spray flow and patter unlike traditional aerosol spray systems, which vary in spraying amounts when tilted. Said property of the valve system apparatus enables it to be used in applying first aid solutions to unusual or difficult wound instances such as accidents involving critically pinned persons, or accidents requiring precise movements and handling. Hence, the valve system of the present invention provides an overall improved efficiency of wound management, which is superior to that of the prior art. Additionally, aerated compositions produced from said valve system delivers a cooling effect to the wound, thereby providing further relief from pain being felt by a wounded subject.

In one embodiment, said valve system used comprises a bag-on-valve system (BoV). In an embodiment wherein said valve system comprises a BoV, system, said BoV comprises a bag attached to a valve. Said bag attached to a valve is folded, rolled, or compressed having no material contained therein which is configured to receive any liquid or fluid material entering the bag through the valve.

In another embodiment of the invention, a method for producing a valve system apparatus for wound management is provided. Said method comprising the steps of placing a bag-on-valve (BoV) inside a storage canister, crimping the BoV onto the storage canister followed by filling said storage canister with propellant gas to obtain a pressurized storage container, and filling the bag of the BoV with a antimicrobial composition via the valve of the BoV.

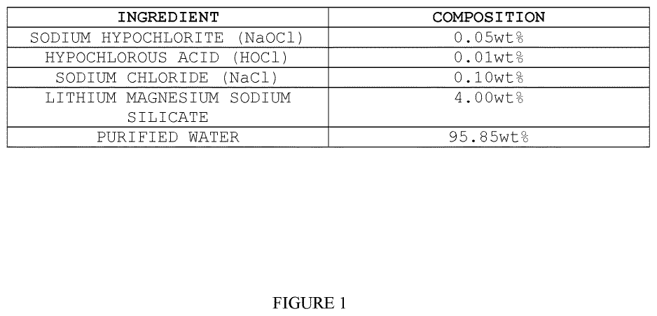

With reference to FIG. 1, FIG. 1 depicts a table illustrating the composition of each ingredient of the antimicrobial composition, in accordance with one particular embodiment. The antimicrobial composition of the present technology has antimicrobial properties. In an embodiment, as shown in the table in FIG. 1, in the antimicrobial composition, the sodium hypochlorite is 0.05 wt %, the hypochlorous acid (HOCl) is 0.01 wt %, the sodium chloride is 0.10 wt % and the synthetic silicate clay based thickening agent is 4.0 wt %, and purified water is 95.85 wt %. In an embodiment, the synthetic silicate clay based thickening agent includes lithium magnesium sodium silicate. In an embodiment, the composition of lithium magnesium sodium silicate is 4.0 wt %.

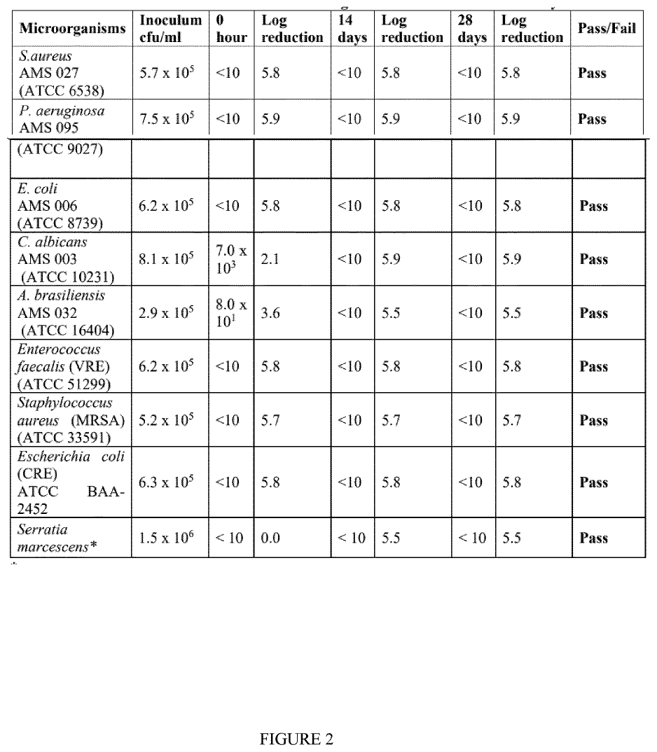

FIG. 2 depicts test results of preservative test according to USP (United States Pharmacopeia) preservative efficacy test, in accordance with an exemplary scenario. The composition of the antimicrobial composition of the present technology was tested for USP 51 and preservative efficacy test by an accredited lab to demonstrate the bacteriostatic effect. The table of FIG. 2 depicts the test result of preservative test of the composition. As can be observed from the table of FIG. 2, the bacteriostatic properties were given inter alia by addition of sodium hypochlorite, which cause biosynthesis alteration in cellular metabolisms and inhibit the growth of bacteria on the acting as preservative. The effect on microbial population reduction has been proved in vitro for clinically relevant strains known to cause wound infection of both gram positive and gram negative as for fungi.

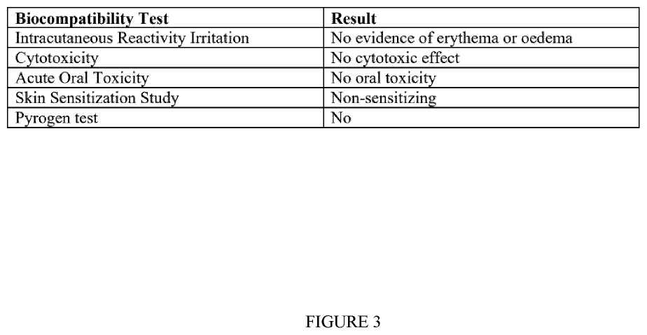

FIG. 3 depicts test results of biocompatibility of the antimicrobial composition of the present technology as per International Organization for Standardization (ISO) 10993 for in vivo and in vitro studies, in accordance with an exemplary scenario. As shown in FIG. 3, there is no evidence of erythema or oedema during intracutaneous reactivity irritation test on the antimicrobial composition of the present technology. In addition, there is no cytotoxic effect or oral toxicity by the antimicrobial composition of the present technology.

FIG. 4 is a flow diagram depicting the steps involved in a particular embodiment of the method of manufacturing the antimicrobial composition of the present technology. The method produces about 3 liters of composition. At step 402, the method includes filling 3 liters of purified water into a container. At step 404, the method includes turning on a high shear mixing machine and setting a speed at 2000 rpm. At step 406 includes adding 120 g±lg of lithium magnesium sodium silicate (thickening agent) slowly from the edge of container into the solution. At step 408, the method includes continuously stirring the mixture at 2000 rpm for 30 minutes. At step 410, the method includes adding 30 mL±0.05 mL of sodium hypochlorite solution (4 wt %-8 wt %) slowly from the edge into the mixture and stir for 5 minutes at 5500 rpm. At step 412, the method includes adding 3.0 g±0.1 g of pure dried vacuum (PDV) salt (NaCl) into the mixture. At step 414, the method includes stirring the mixture at 5500 rpm for 15 minutes until the mixture is completely homogenized. In an embodiment, the sodium hypochlorite solution is 4 wt % to 8 wt %.

### EXAMPLES

### Example 1

Example 1 included filling three liters of purified water into a container. The example further included turning on a High Shear Mixing machine and setting a speed at 2000 rpm. The example further included adding 120 g±1 g of Lithium Magnesium Sodium Silicate (thickening agent) slowly from the edge of container into the solution. The example further included continuously stirring the mixture at 2000 rpm for 30 minutes. The example further included adding 30 mL±0.05 mL of sodium hypochlorite solution slowly from the edge into the mixture and stir for 5 minutes at 5500 rpm. The example furthermore included adding 3.0 g±0.1 g of PDV (NaCl) salt into the mixture. The example furthermore included stirring the mixture until the mixture is completely homogenized, wherein the mixture was stirred at 5500 rpm for 15 minutes. The sodium hypochlorite solution was in the range 4 to 8 wt %.

### Example 2

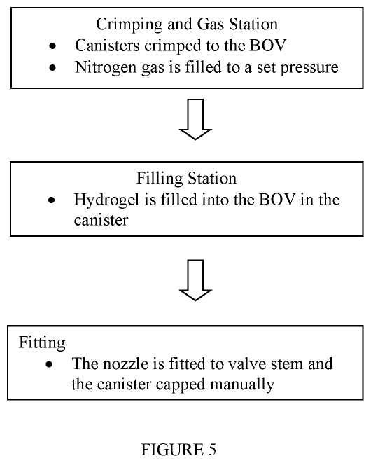

In an example provided which illustrated in FIG. 5, is a semi-automatic crimping, gassing and filling machine (AM-04) was used to fill pressurized storage canisters with form of an antimicrobial composition. First, a bag-on-valve (BoV) was placed inside a storage canister. The valve of the BoV was crimped onto the storage canister followed by filling said storage canister with the propellent gas, nitrogen to obtain a pressurized storage canister comprising a BoV. The bag of the BoV was then filled with the antimicrobial composition via the valve into the bag attached (BOV). This feature allows the product to physically separated from the propellent gas used.

Below are the summary of the process parameter setting for Small (50 g) and Large (100 g) canisters:

| | Crimp | Crimp | | Mass Set., | |
|---|---|---|---|---|---|
| | Depth | width | Propellent | M_{SET} | |
| | Set., CS_{D} | Set., CS_{W} | pressure | (Hand | Air |
| | (Revolution | (Revolution | Set., P_{N} | wheel | Pressure |
| Canister | in Degrees) | in Degrees) | (MPa) | position) | (Mpa) |
| Small | 3420 | 1270 | 0.30 | 35.8 | 0.6 |
| (50 g) | | | | | |
| Large | 3420 | 1270 | 0.60 | 58.5 | 0.6 |
| (100 g) | | | | | |

* 1 turn = 360 degrees

(BOV, non-aerosol) is classified under non-flammable product as per EC Council Directive 75/324/EEC (Annex 1—Definition 1.9).

In an example provided, the main components of this product, particularly the valve system apparatus for wound management are:
antimicrobial wound composition: 0 wt % flammable components (water based).
Nitrogen Gas: Inert gas which contains 0 wt % flammable components. The term ‘Aerosol’ here refers to product nature, which it depends upon the power of pressure to expel the contents from container (U.S. FDA—Tamper Resistance Regulation—21 CFR 700.25).

In one embodiment, the valve apparatus for wound management of the present invention bag-on-valve technology is a compartmentalized aerosol dispenser, comprising a metal/plastic bag attached to the valve or valve body. There are no mixture of propellant gas and product takes place (NIST Guideline on Aerosol & Bag-On-Valve Definition (DDF Jan. 28, 2012)—February 2012).

Various embodiments of the present invention provide an antimicrobial composition developed for moistening and debridement of acute and chronic wounds such as stage I-IV ulcers, venous stasis and diabetic ulcerations, post-surgery wounds, first and second-degree burns, and bedsores. s help maintain a moist wound environment, promote granulation and epithelialization, and facilitate autolytic debridement.

### Example 3

In an example provided which is illustrated in FIG. 2, Preservative efficacy test USP 51 were used to evaluate antimicrobial properties of gel when tested against selected bacteria, fungi and yeast respectively

In this method, the gel is inoculated with a controlled quantity of specific microorganisms. The test then compares the initial level of microorganisms to the test samples at various time intervals over a period of 28 days at a specified temperature. A logarithmic reduction of organisms is evaluated at prescribed time interval to quantitatively evaluate the effectiveness of the antimicrobial properties of gel to prevent microbial proliferation and/or kill or reduce the organism's population

For Topical Products, the preservative is effective in the product examined if:

Bacteria—Not less than 2.0 log reduction from initial count at 14 days and no increase from the 14 days' count at 28 days is observed for bacteria (Escherichia coli, Staphylococcus aureus, Pseudomonas aeruginosa and Burkholderia cepacia)

Yeast and Mould—No increase from the initial calculated count at 14 and 28 days observed for yeast and mould (Candida albicans and Aspergillus brasiliensis)

### Example 4

In an example provided, an iodometric titration was performed for the determination of hypochlorous acid and sodium hypochlorite in the raw material used to in preparing the hydrogel composition of the present invention.

Initially the reagents used were prepared according to the following procedures:

| Reagent | Preparation |
|---|---|
| 0.2N sodium | Weigh 4.96 grams of Na₂S₂O₃•5H₂O |
| thiosulfate solution | into a 100 ml volumetric flask; |
| (Na₂S₂O₃•5H₂O) | add distilled water up to the mark |
| | and mix it to dissolve. |
| 0.05N Sodium | Weigh 1.24 grams of Na2S2O3•5H2O |
| thiosulfate solution | into a 100 ml volumetric flask; |
| (Na₂S₂O₃•5H₂O) | add distilled water up to the mark |
| | and mix it to dissolve. |
| 10% Potassium | Weigh 10 grams of KI and dissolve |
| Iodide solution | it in 100 mL of distilled water. |
| Starch | Weigh 0.5 grams of starch and |
| | dissolve it in 100 mL of distilled |
| | water. Heat up the mixture (40° C.) |
| | and stir it well until the |
| | solution turns almost clear and |
| | produces slight condensation. Cool |
| | the starch solution down to room |
| | temperature before using it. |
| Acetic acid | Measure 35 mL of acetic acid using |
| (glacial) 100% | measuring cylinder and transfer it |
| | into a 250 mL conical flask. |

The samples prepared were sodium hypochiorite, antimicrobial aqueous solution (Hydrocyn® aqua, which is identical to Bactiguard® Wound Care solution), and antimicrobial gel composition (Hydrocyn® aqua gel, which is identical to Bactiguard® Wound Care gel with the content according to the table in FIG. 1 and falling under claim 1) of the present invention according to the following preparations:

| Samples | Preparation |
|---|---|
| Raw material Sodium Hypochlorite | 1 ml of sample was pipetted into a |
| (10%) | 100 ml volumetric flask and filled |
| | with distilled water up to the |
| | mark. The solution prepared was |
| | transferred to a 250 ml conical |
| | flask. |
| antimicrobial aqueous solution | 100 ml of sample was transferred |
| (Hydrocyn ® aqua, which is a wound | on to a 250 ml conical flask |
| care solution from Bactiguard ®) | |
| antimicrobial gel composition | 5 grams of sample was weighed |
| (Hydrocyn ® aqua gel, which is a | and was dissolved in 100 ml of |
| would care gel from Bactiguard ®, | distilled water, then transferred |
| see the table in FIG. 1 for the | to a 250 ml conical flask. |
| content) | |

The samples were subjected to titration. A burette was rinsed with few amounts of sodium thiosulfate solution then was filled with sodium thiosulfate just above the 0 mark. A small amount of the solution was let out through the burette tip to release trapped bubbles. About 25 ml of 10% potassium iodide solution into the sample solution. About 10 ml of acetic acid was added to bring about acidic condition. The addition of the acetic acid and potassium iodide causes the sample solution to change color from colorless to amber brown. Then, titration was performed onto the sample solution using 0.2N/0.05N sodium thiosulfate to a straw yellow color. Titration was done quickly as iodine liberates quickly. About 5 ml of starch indicator was added and titration was continued until blue color disappears. The added starch indicator reacts with iodine to form a very intense blue/purple color complex. The titration was stopped as soon as a clear solution was obtained. Then, the volume of sodium solution titrated was recorded for further calculation.

The number of moles of sodium thiosulfate for each titration was calculation using the equation

$$Moles of {Na}_{2} {S}_{2} {O}_{3} = ( molarity of {Na}_{2} {S}_{2} {O}_{3} ) \times ( volume of {Na}_{2} {S}_{2} {O}_{3} ) = \frac{0.2 N \times titrated volume ( ml )}{1000}$$

and through the calculated moles of sodium thiosulfate, the stoichiometry reaction was used for calculating the moles of hypochlorite

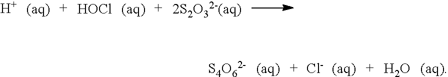

The stoichiometry of the equation shows that there are 2 moles of thiosulfate ion per mole of hypochlorous acid:

$$Mole of HOCl = \frac{1}{2} moles of {S}_{2} {O}_{3}^{2 -}$$

$$Mole of HOCl = Moles of NaOCl .$$

The concentration (% by weight) of NaOCl in the raw material was calculated using the following computation:

$$\% NaOCL in sample = \frac{Mass of NaOCl ( X )}{Mass of sample ( Y )} \times 100 = \frac{( 0.05 ) ( V ) ( N ) ( 74.44 g \cdot mol )}{M} \times 100$$

The V is the volume of the titrant (in ml), N for the normality of sodium thiosulfate used (N), and M for the mass of the sample. The calculated mw of NaOCl is 74.44 g/mol.

The concentration (% by weight) of NaOCl and HOCl in the antimicrobial aqueous solution (Hydrocyn® aqua, which is a wound care solution from Bactiguard®) was calculated using the following computation:

$$\% NaOCL in sample = \frac{Mass of NaOCl ( X )}{Mass of sample ( Y )} \times 100$$

$$\% NaOCL in sample = \frac{Mole of NaOCl \times 74.44 g / mol}{100} \times 100$$

$$\% HOCL in sample = \frac{Mass of HOCl ( X )}{Mass of sample ( Y )} \times 100$$

$$\% HOCL in sample = \frac{Mole of NaOCl \times 52.46 g / mol}{100} \times 100$$

In the provided calculations, the normality of sodium of thiosulfate used was 0.05N, mw of NaOCl is 74.44 g/mol, and the mw of HOCl is 52.46 g/mol.

As will be readily apparent to those skilled in the art, the present invention may easily be produced in other specific forms without departing from its essential characteristics.

The present embodiments are, therefore, to be considered as merely illustrative and not restrictive, the scope of the invention being indicated by the claims rather than the foregoing description, and all changes which come within therefore intended to be embraced therein.

### Example 5

Since the water quality of the water used for the manufacture of the composition is of great importance, an example of the water purification is given for the water to be used when making the composition.

First, tap water was fed into a first stage of a RO/DI system, which consists of a Sediment filter (mainly comprises various sizes of sands) and a Carbon filter. The Sediment filter was used to trap the large free floating particles or contaminants. Whereas the Carbon filter consisted of granulated carbon and absorbed organics and other dissolved contaminates such as chlorine and chloramines.

After the carbon filtering stage, the water entered the softener column and the hard mineral ions (such as magnesium, Mg²⁺ and calcium, Ca²⁺) in the water were removed. When the hard water entered into the mineral tank, it flows through a bed of spherical resin beads. These beads are charged with a sodium ion. The resin beads are anions with negative charge. The calcium and magnesium minerals have a positive charge, making them cations. As the hard water passed through the resin, the beads grabbed hold of the mineral ions and removed them from the water. When the bead seized the mineral ions, sodium ions were released.

Subsequently, water was fed to a Reverse Osmosis (RO) system and filtered through multiple layers of thin film that removed a majority of contaminates such as salts, bacteria, heavy metals, and other organics. From here, the water was split into two different water lines: a waste water line and a product water line. The product water was almost pure and travelled into the storage tank that acted as a reservoir for the DI (deionization) system. Whereas certain percentage of the waste water was re-circulated into the RO system and the remaining part will be discharged to a drainage line.

Prior to feeding into DI system, the RO water was chilled to a temperature range of 20-25° C. by a chiller. The chilled water passed through an Ultra-violet (UV) purifier to go through a disinfection process. The UV purifier exposed living organisms, such as bacteria, viruses, or cysts (like Cryptosporidium and Giardia) to a germicidal ultraviolet radiation to disrupt the DNA in pathogenic microorganisms, so they at least cannot reproduce.

After UV disinfection the water passed through three identical deionizing columns comprising a resin. At the deionization stage, there was used an ion exchange process that attracts mineral impurities such as sodium and other metallic elements. Negatively charged Cation resin will attract positively charged ions in the water, while positively charged Anion resin attracts the negative ions. Finally, the DI water passed through ultrafine filter cartridge (i.e. 0.45 μm and 0.2 μm), then the water was fed in tubes to points-of-use in the production floor. The unused purified water was circulated back to the storage tank and fed into the DI system again to repeat the same deionization process continuously.

1. An antimicrobial composition, comprising (OCI)− ions in an amount in the range 0.03-0.2 wt % with counterions Na+ ions in an amount of 0.005-0.05 wt % and H+, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity β measured at pH 8.0 is lower than 0.10.

2. The antimicrobial composition according to claim 1, wherein the amount of sodium hypochlorite is in the range 0.04-0.06 wt %.

3. The antimicrobial composition according claim 1, wherein the amount of hypochlorous acid (HOCl) is in the range 0.005-0.02 wt %.

4. The antimicrobial composition according to claim 1, wherein the amount of sodium chloride is in the range 0.05-0.2 wt %.

5. The antimicrobial composition according to claim 1, wherein the synthetic silicate clay based thickening agent comprises lithium magnesium sodium silicate.

6. The antimicrobial composition according to claim 1, wherein the synthetic silicate clay based thickening agent is present in an amount in the interval 3-5 wt %.

7. The antimicrobial composition according to claim 1, wherein purified water constitutes the remaining part of the composition in addition to sodium hypochlorite (NaOCl), hypochlorous acid (HOCl), synthetic silicate clay based thickening agent, and sodium chloride (NaCl).

8. The antimicrobial composition according to claim 1, wherein said composition has a free available chlorine in the range 30-200 ppm.

9. A method for manufacturing an antimicrobial composition, the method comprising the steps of:
a) providing
i. purified water, wherein the purified water before addition to the composition has a hardness measured according to ISO 15923-2:2017 of less than 5 ppm and a resistivity measured according to ISO 3696:1987 of more than 15 MΩ/cm;
ii. a synthetic silicate clay based thickening agent;
iii. a sodium hypochlorite solution, and iv. sodium chloride, and
b) mixing the purified water, the synthetic silicate clay based thickening agent, the sodium hypochlorite solution, and the sodium chloride to obtain the antimicrobial composition,
wherein the composition has a pH in the interval 7.5-10.0, and wherein the buffer capacity p measured at pH 8.0 is lower than 0.10.

10. The method according to claim 9, wherein the sodium hypochlorite solution has a concentration in the range 4-8 wt %.

11. The method according to claim 9, wherein the synthetic silicate clay based thickening agent comprises lithium magnesium sodium silicate.

12. A valve system apparatus for wound management, said valve system apparatus comprising,
a bag-on-valve (BoV) inside a pressurized storage canister; and
the antimicrobial composition of claim 1 for wound management contained in said bag.

13. The valve system apparatus for wound management of claim 12, wherein the pressurized storage canister comprises a non-flammable propellant.

14. The valve system apparatus for wound management according to claim 12, wherein said pressurized storage canister is an aerosol spray canister.

15. A method for producing a valve system apparatus for wound management comprising steps of:
a) placing a bag-on-valve (BoV) inside a storage canister;
b) crimping the BoV onto the storage canister followed by filling said storage canister with propellant gas to obtain a pressurized storage container; and
c) filling the bag of the BoV with the antimicrobial composition according to claim 1 via the valve of the BoV.

16. A method for delivery of an antimicrobial composition to a wound, said method comprising applying the antimicrobial composition of claim 1 to a wound.

17. The method according to claim 16, wherein the applying of the antimicrobial composition to the wound is made from a valve system apparatus, said valve system apparatus comprising a bag inside a pressurized storage canister and the antimicrobial composition for wound management contained in said bag.

18. A method for the treatment of a wound, comprising applying to the wound the antimicrobial composition of claim 1.

19. A composition for prevention or treatment of infections in wounds, comprising (OCI)− ions in an amount in the range 0.03-0.2 wt % with counterions Na+ ions in an amount of 0.005-0.05 wt % and H+, synthetic silicate clay based thickening agent in an amount in the range 1-10 wt %, sodium chloride (NaCl) in an amount in the range 0.03-0.3 wt % and purified water, wherein the purified water before addition to the composition has a hardness measured according to ISO 15923-2:2017 of less than 5 ppm and a resistivity measured according to ISO 3696:1987 of more than 15 MΩ/cm, wherein the composition has a pH in the interval 7.5-10.0, and wherein the buffer capacity p measured at pH 8.0 is lower than 0.10.
